(19) United States
(12) Patent Application Publication
(10) Pub. No.: US 2021/0023351 A1
(43) Pub. Date: Jan. 28, 2021

(54) MODULAR BUILT MEDICAL DEVICE AND PROCESS FOR ASSISTING INTERMITTENT CLEAN SELF-CATHETERIZATION AND/OR SELF-INSTILLATION OF DRUG-SOLUTIONS INTO THE BLADDER IN FEMALE PATIENTS

(71) Applicants: Sándor LOVÁSZ, Budapest (HU); Gábbor RÉNYI, Budapest (HU); János GIBER, Budapest (HU)

(72) Inventors: Sándor LOVÁSZ, Budapest (HU); Gábbor RÉNYI, Budapest (HU); János GIBER, Budapest (HU)

(21) Appl. No.: 16/074,640

(22) PCT Filed: Feb. 1, 2017
(86) PCT No.: PCT/HU2017/000006

§ 371 Date: Aug. 1, 2018

(30) Foreign Application Priority Data
Feb. 1, 2016 (HU) P1600050

(51) Int. Cl. A61M 31/00; A61M 3/02

(52) U.S. Cl. CPC A61M 31/00; A61M 3/0262; A61M 2205/586; A61M 2025/0166; A61M 2205/583; A61M 2210/1085; A61M 2210/1092; A61M 2205/3306

(57) ABSTRACT

The subject matter of the invention is a medical device for assisting self-catheterization and instillation of drug-solutions into the bladder in female patients, consisting of switchable light force (6), of power supply (7), of mirror (8) and/or of one or more cameras (13). The device can be fixed on the dosing syringe (1) itself and beside or instead of the mirror (8) is consisting also of one or more cameras (13). The catheter (2) and the urine drainage bag (13) is connected through a three-way tap (9). A holder element (12) is fixed on the grip element (11). On or in the holder element (12) a micro switch (5), a light source (6), power supply (7) can be found. Further on a mirror (8) or a camera (13) is fixed expediently to the holder element (12) by a fixation element (15).

The subject matter of the invention is a medical device for assisting self-catheterization and instillation of drug-solutions into the bladder in female patients, consisting of a grip element advantageously a syringe, or advantageously a handle element replacing the syringe, further on of a unit releasable connectable to the bottom side of the handle near the catheter, and releasable clip-on connectable also to the catheter, advantageously of a spring-grabbing clip-on unit suitable for grabbing the catheter safely or further on of a three-way tap replacing the connecting unit, and further on of a catheter, advantageously of a 15 to 25 cm long female catheter, further on of an urine drainage bag, further on of a switchable light source, further on of a power supply and further on of a mirror if necessary.

The handle is advantageously hollow with the right thickness of wall for the proper stability and having the proper shape and size for grabbing it with hand, advantageously cylindrical and closed on the upper side or further on advantageously solid, and further on advantageously a body with the same diameter and length of a commercially available syringe.

Further on the subject matter of our invention is process for use of the device according to our invention for assisting self-catheterization and self-instillation of drug-solutions into the bladder in female patients.

There are thousands of patients having urinary retention or having problems with bladder drainage because of nerve injury to the spinal cord or after difficulty urination the high pressure and quantity of the residual urine are endangering the kidney function and/or causing an intractable urinary tract infection.

The multiple nerve injury generated during radical pelvic surgeries used because of the cancer processes of the pelvic tract (colon cancer, uterine tumors, ovarian cancer) is also another frequent reason of ischuria to be treated.

In such cases earlier the patients were wearing a so called “permanent” balloon-catheter for a whole life-time with 2-6 weeks changes, depending on the material of the catheter.

For this traditional solution the patients should be transported to the surgeries or supplied at home, and this is a real great burden on healthcare.

Wearing a permanent catheter causes continuous irritation, pains, bleeding in urine and so serious deterioration in the quality of life.

More serious complications like chronic bacterial urinary tract infections, and thus resulting inflammation of the renal pelvis, or purulent inflammation of urethra with high fever could influence even the life expectancy of the patients disadvantageously

In the last decades a professionally recommended and more and more widespread method got known, the so called Intermittent Clean Self-Catheterization performed by the patients by their own.

The insertion of the catheter into the external urethral orifice (urinary meatus, meatal opening) and the drainage of the bladder is performed mostly by the patient sometimes using external assistance.

There are lot of difficulties and disadvantages of the clean self-catheterization in female patients. For the exploration of the external urethral orifice and the insertion of the catheter a high manual dexterity and motility is needed by the female patients.

Therefore many patients are not capable for self-catheterization by their own, only by using external assistance. Keeping the proper sterility is very difficult, almost impossible using the method of permanent self-catheterization in the presence form.

For the significant part of the previous group of patients regular local medication of the bladder is needed to treat chronic bacterial inflammation, bleeding or other associative bladder diseases.

By using the known, traditional self-catheterization method these kind of treatments are hard to carry out, because the syringe (20 cm) connecting to the drain-catheter (20-30 cm) is practically in unattainable distance from the patient.

By the third group of the patients no bladder drainage is needed, because they are capable for spontaneous drainage, but because of their basic diseases regular local medication of the bladder is needed.

The basic diseases are the complications following the radiotherapy of the cancer of the pelvic tract (radiation injury and tissue damage of a part of the bladder, mostly the base of the bladder or the urethra with pang, bleeding and serious micturition).

The most effective treatment of these diseases is the instillation of bladder with drug solutions (GAG layer remounts and regenerators) and used by the medication of Bladder Pain Syndrome/Interstitialis Cystitis (BPS/IC).

At serious cases the injured of the palliative radiation without having any hope for completely healed are also added to this group of patients.

During pelvic radiations concerning other indications (adjuvant or palliative (in case of advanced diseases) treatments after surgical removal of bladder, colon, uterine and ovarian tumors) some parts of the bladder could be significantly radiated, therefore intravesical bladder instillation is needed.

BPS/IC patients living far from a medical assistance cannot be easily treated regularly because of the long journeys.

Clean self-treatment in this group has the advantage that, the symptoms could be treated immediately with bladder instillations in the worse stages of the disease, there is no need to wait for a medication by the doctor.

The number of female BPS/IC patients is about 20.000, although the number of diagnosed cases are only fraction of the real value because of the bad discovery rate of the disease BPS/IC.

In regard to the fact that, the professional interest to the BPS/IC is growing worldwide more and more, the detection rate of the BPS/IC and the number of BPS/IC patients will also sky-rocket.

BPS/IC patients can be treated in about 100% by a catheter-free bladder instillation by using a 16 syringe adapter for injection, protected by a patent application (Hungarian application number is P1500648).

Chronic bacterial bladder inflammations existing for years can be detected more and more often caused by the pathogens being multi-resistant against all antibiotics.

In such cases the regeneration of the defending mucous-layer (glycosaminoglycan, shortly GAG layer) on the surface of the inner wall of the bladder by regular local bladder instillations is almost the only effective treatment.

Benefits of the clean self-catheterization could be enjoyed by all these groups of patients if bladder instillation could be performed by their own.

Difficulties, disadvantages of catheter-free self-bladder instillation are the same as of clean self-catheterization disclosed above.

Therefore the medical device according to our invention consists mostly of the same elements, only some parts are different according to the special needs of the three different groups of patients.

For the self-catheterization in female patients many solutions could be found earlier.

A device for self-catheterization in female patients with tripod is disclosed by the patent application US 2015062729. The tripod is equipped with a movable mirror equipped with a light source with battery. The mirror is adjustable in different planes.

With the assistance of the mirror and the light source the device can be adjusted to the direction of the urethra.

With the assistance of the device the urinary meatus can be seen well by the women, assisting the self-catheterization in this way.

A large-scale, complicated device is disclosed in this invention where either the installation, or the handling or the storage of the device is complicated and cumbersome.

Further on the proper emplacement for the patient and the device is also cumbersome to find.

A device for self-catheterization for women in case of not proper bladder drainage is disclosed in the U.S. Pat. No. 5,084,036 patent application.

The device consists of a basic part for the insertion into the vagina, of a support part being perpendicular to the basic part, which can be found on the opposite of the outer part of the vagina, and of a grip element which is part of the basic part.

A transfer tube is connected to the basic part: one end of the tube is in front of the outer part of the vaginal opening (urinary meatus), and the other end of the tube can be connected to the end of the catheter for insertion, directed into the urethra, making the self-catheterization easier.

Similar to the previous solution, the device according to this invention is also large-scaled, complicated, it is difficult to be handled, and the storage and installation is also cumbersome.

A device for insertion of the catheter and self-catheterization in female patients is disclosed in the patent document DE 10060088 consisting of a holder part for insertion into the vaginal opening and of a front leading part for the catheter.

The front leading part and the holder part is placed in a distance appropriate to the distance between the vagina and the urethra with a spacer. The holder part is conic shaped and its intersection is increasing while moving away from the spacer. The front leading part is releasable connected to the spacer and its intersection gets narrower to the direction to the spacer.

This solution is very simple, but it is not suitable for ensuring attainment of our objective.

The common disadvantage of the earlier known solutions of the prior art is that, all subject matter of the inventions can only solve the problems of urine drainage with the earlier disclosed handicaps.

Our target was to develop a device which can be used by all groups of patients, the handling is easy and also easy for learning for all patients by proper using of simple accessories.

Further on our target was to find a solution for treatment, nursing and supplying of patients in serious condition with wrong quality of life, suffering in chronic diseases or living with disabilities, reducing significantly the number of complications of the catheterization and the burden of such patients and to improve their life expectancy It was recognised that, on one hand the facilitating elements for the self-catheterization should be fitted together and on the other hand the device itself should be used without using any further accessories.

Further on in addition according to our invention the device could be installed according to the own needs of all group of patients.

This makes possible to create a simpler and better usable device for self-catheterization.

It is enough to position properly the lightweight and easily positionable device according to our invention under visual control. The position of the patient should not be adjusted to the device.

As with the assistance of the device the catheter can be inserted into the urethra one handed by the patient, the vulva can be explored properly and so the urinary meatus can be located and catheterized easily, therefore a higher level of sterility can be assured.

Thus the subject matter of our invention is medical device for assisting intermittent clean self-catheterization and self-instillation of drug-solutions into the bladder in female patients

The device consists of a grip element advantageously of a syringe, or a handle element, and of a unit releasable connectable both to the grip element and to the catheter, advantageously of a spring-grabbing clip-on unit or of a three-way tap, and further on of a catheter, further on of an urine drainage bag, further on of a switchable light source, further on of a power supply and further on of a mirror if necessary. Instead of or beside the mirror the device consists of a camera.

The catheter and the urine drainage bag is connected through the three-way tap so that, the syringe connected to the medium outlet of the three-way tap serves also as the grip-element of the device or in case of the first group of patients the catheter is fixed simply to the bottom side of the handle and leads directly to the urine drainage bag.

In case of the first group of patients where the target is solely the drainage of the bladder advantageously the already disclosed handle is used as grip element instead of the syringe and the three-way tap is replaced by the connecting spring-grabbing clip-on unit, clipping it simply to the catheter, fixing the catheter this way to the bottom side of the handle element.

Additionally there is a holder element fixed to the grip element.

The micro-switch, the light source and the power supply are fixed on and/or in the holder element.

The mirror and/or the camera are (is) fixed expediently to the holder element.

The camera could be wired or wireless and is designed suitable for connection to any receiving set.

The subject matter of our invention is further on process for use of a medical device assisting clean self-catheterization and self-instillation of drug-solutions into the bladder in female patients where the device according to our invention is used.

According to the process the grip element, advantageously the syringe or the handle element consisting of the holder element and connected to the catheter is taken by one hand, the end of the leakage-free adapter or of the catheter and the surroundings of it is lighted and placed near to the urinary meatus. Next the mirror and/or the camera will be placed so that, the picture shown in the mirror or in the camera shows the end of the catheter or the adapter and the urinary meatus at once, and so the adapter or the catheter can be inserted easily into the urinary meatus in a proper length and the urine drainage and/or the bladder drug instillation can be performed under the patient's own hand.

Further advantageous solutions of our invention are disclosed by the specification and by the claims.

The subject matter of the invention will be disclosed according to the figures in details.

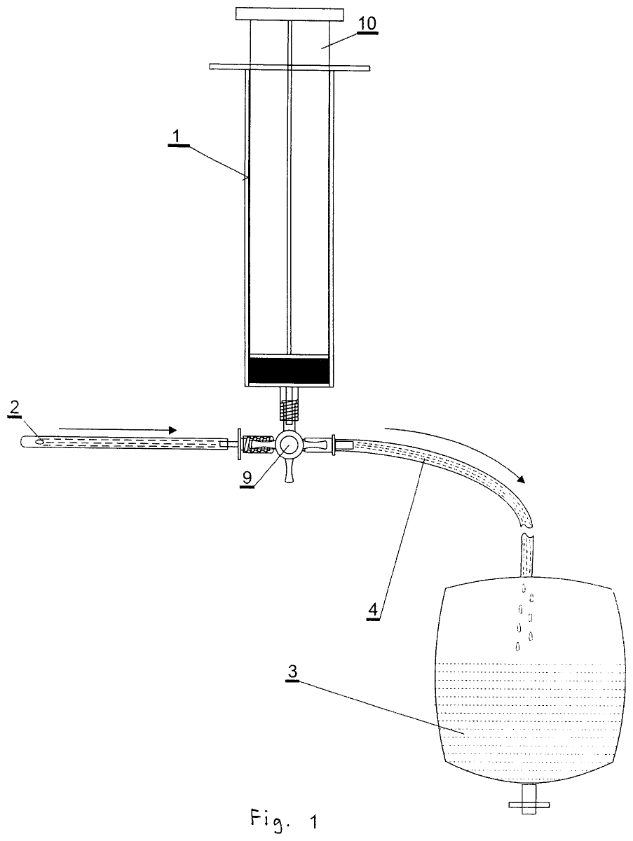

The process of the simple drainage is shown on the FIG. 1 in a side view.

The device according to the invention consist of a 2 female catheter with a proper but shorter than usual length appropriate to the shorter female urethra.

The 9 three-way tap is positioned to a straight flow. The 4 tube of the 3 urine drainage bag is connected to the tap, making an urine drainage in closed system possible.

In this case the 1 syringe connected to the medium outlet of the three-way tap has only a grip-element, leader function

The 12 holder element according to our invention placed on the 1 syringe is not shown in FIG. 1 yet.

As an advantageous solution of the invention in case of simple drainage the 1 syringe is replaced by the 1 handle as grip element and the 9 three-way tap is replaced by the connecting 9 spring-grabbing clip-on unit. The catheter releasable connected advantageously clip-on fixed to the bottom side of 1 handle can be leaded directly to the 3 urine drainage bag.

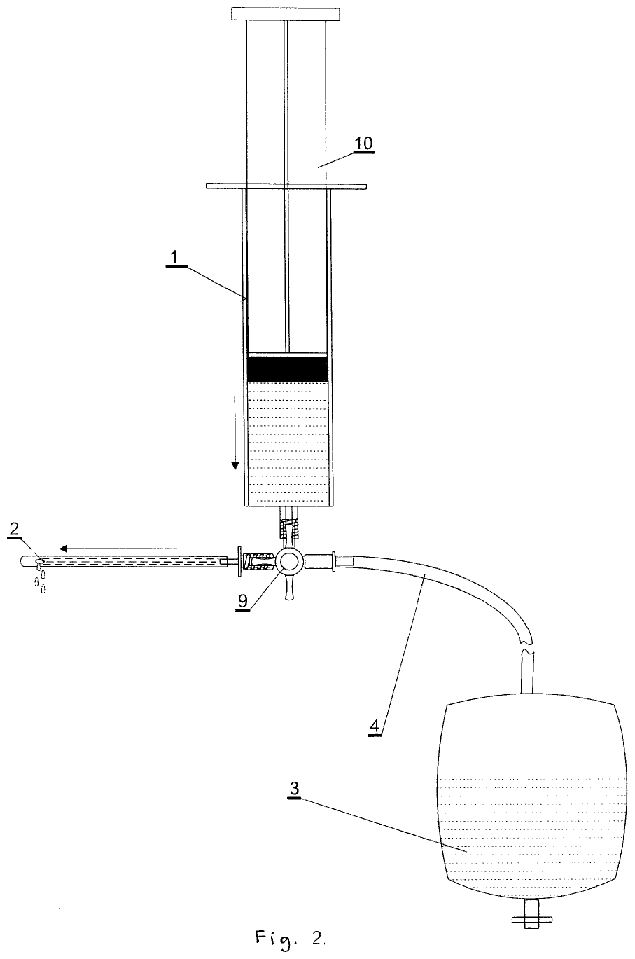

According to the side view of the FIG. 2 the device consist of 2 female catheter with a proper but shorter than usual length where after urine drainage also the drug instillation could be performed.

After changing the position of the 9 three-way tap, by pressing the 10 piston of 1 syringe the drug solution can be injected into the bladder through the 2 catheter.

The 12 holder element according to our invention placed on the 1 syringe is not shown in FIG. 2 yet.

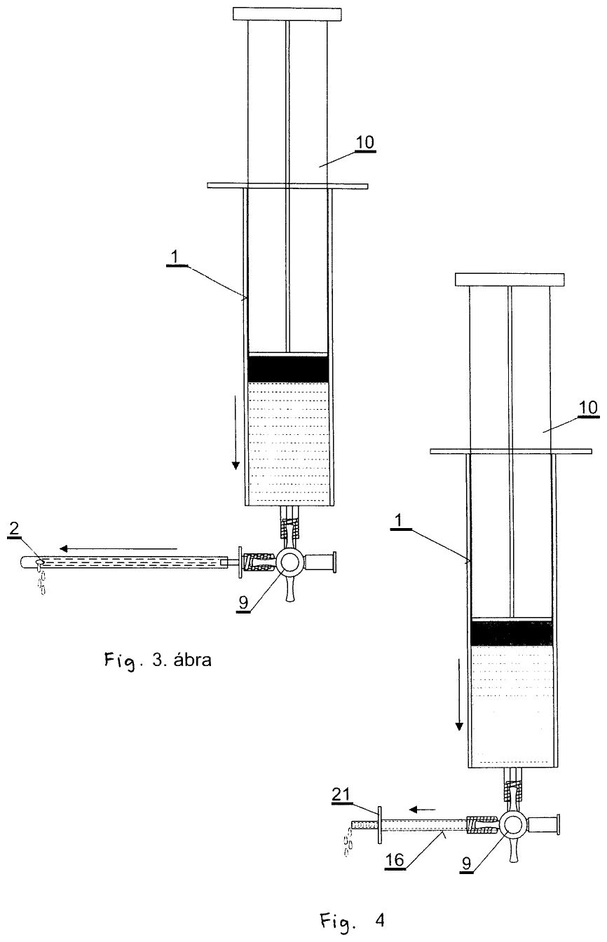

As illustrated in the side-view of FIG. 3 a possible method of drug instillation is shown using a shorter than usual 2 catheter where no urine drainage of the bladder is needed, so the 3 urine drainage bag is not connected to the system.

In FIG. 4 a possible way of drug instillation is shown, using the 16 leakage-free adapter for injection, where no bladder drainage is needed, so the 3 urine drainage bag is not connected to the system

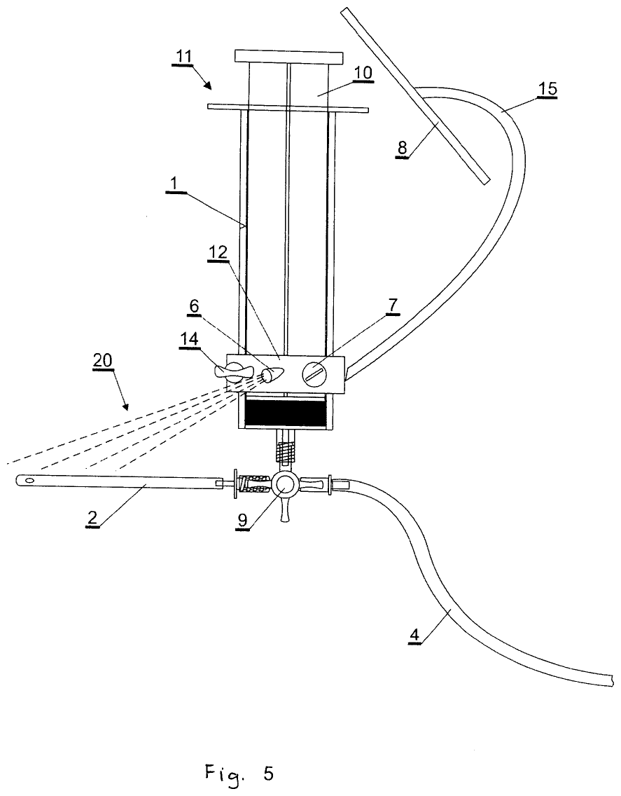

The side view of the device for drainage consisting of 2 catheter and of 12 holder element according to our invention is shown in FIG. 5, where the 8 mirror is fixed to the 12 holder element by a 15 flexible self-holding arm.

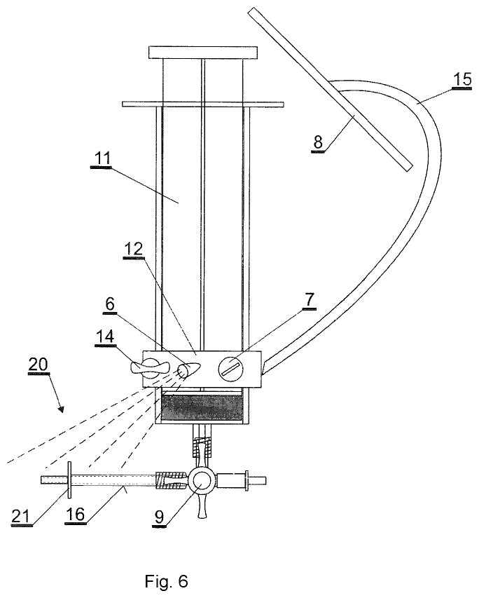

The side view of the injecting device consisting of 12 holder element and of 16 leakage-free adapter for drug instillation is shown in FIG. 6 where the 8 mirror is fixed to the 12 holder element by a 15 flexible self-holding arm.

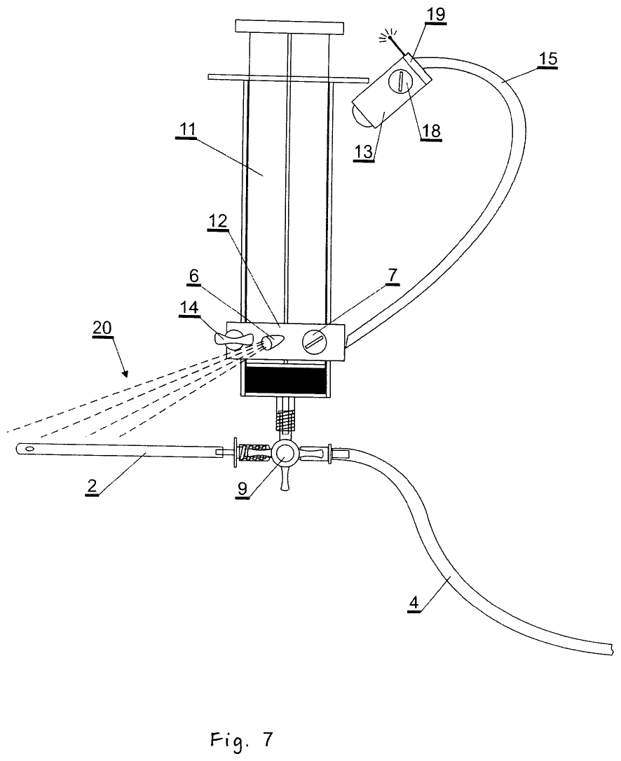

FIG. 7 is the side view of the device consisting of 12 holder element and of 2 catheter where 13 camera is fixed to the 12 holder element.

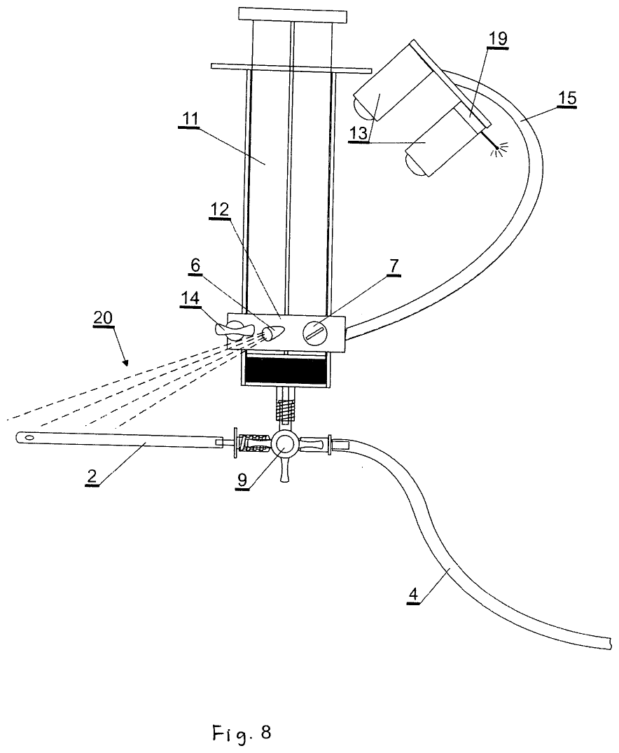

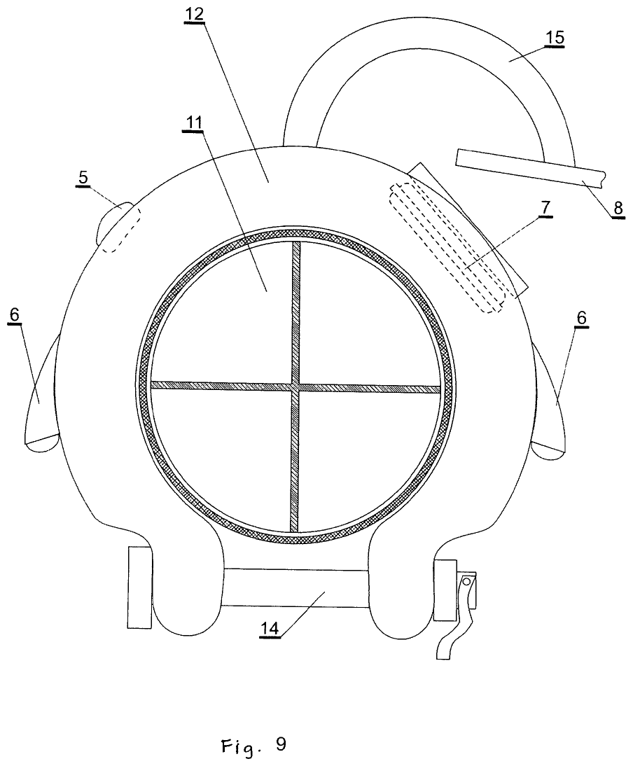

FIG. 8 is the side view of the device consisting of 12 holder element and of 2 catheter where more than one, advantageously two pieces of 13 camera suitable for stereo-recording is fixed to the 12 holder element.

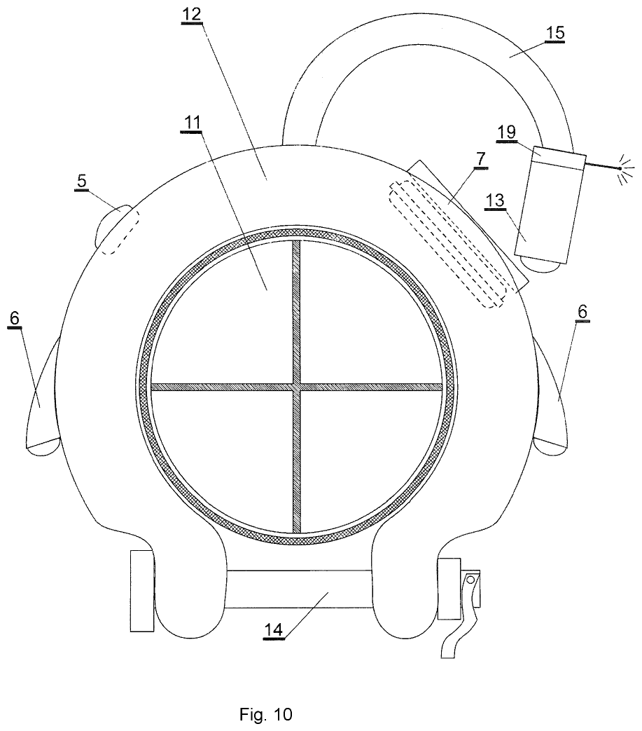

FIG. 9 is the top view of the 12 holder element consisting of 8 mirror fixed to the 11 grip element where the 11 grip element is shown in section.

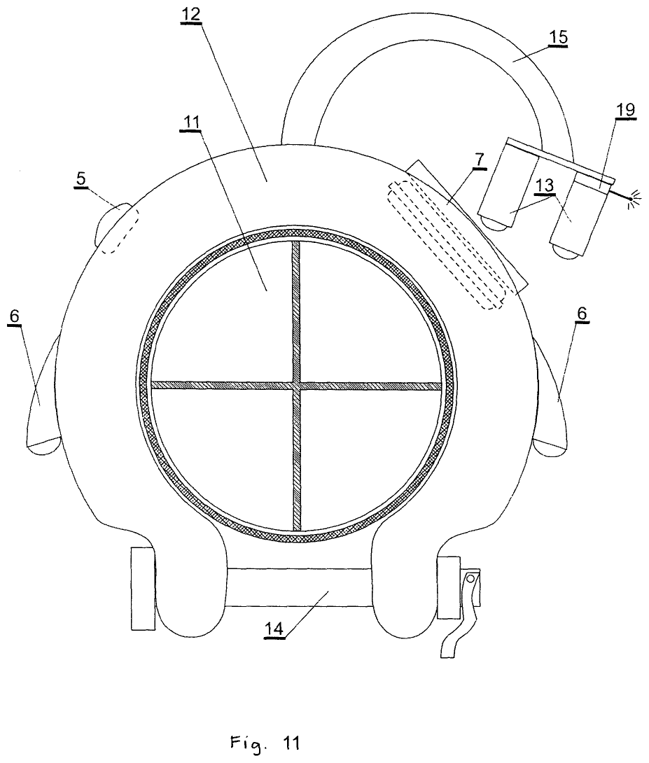

FIG. 10 is the top view of the 12 holder element according to our invention consisting of 13 camera, fixed to the 11 grip element where the 11 grip element is shown in section.

FIG. 11 is the top view of the 12 holder element according to our invention consisting of 13 stereo-camera, fixed to the 11 grip element where the 11 grip element is shown in section

The medical device according to our invention for assisting clean self-catheterization was developed for female patients by further developing the earlier known, and used device to make the self-catheterization easier for the patients.

The device for self-catheterization shown in FIG. 1 can be used by the patients who have ischuria and therefore having significant quantity of residual urine.

In this case the device consists of 1 syringe, 2 catheter, and 3 urine drainage bag consisting of 4 tube. All these elements are connected to the 9 three-way tap consisting of Luer Lock connectors. This connection makes possible the urine drainage through the 2 catheter and 4 tube of 3 urine drainage bag into the 3 urine drainage bag.

As an advantageous solution of the invention by this group of patients the 1 syringe is replaced by 1 handle, the 9 three-way tap is replaced by 9 connecting spring-grabbing clip-on unit. In this case the 1 handle is fixed by using the 9 clip-on unit directly to 2 catheter and the 2 catheter is connected to the 4 conic shaped tube of the 3 urine drainage bag, leading the urine directly into the 3 bag through the 2 catheter.

The most common application of our invention is the simple bladder drainage by self-catheterization.

For this application as an advantageous solution of the invention the commercially available, low priced single use female catheter with approx. 20 cm length can be used.

The 3 drainage bag is connected to this catheter through the 4 tube of the 3 urine drainage bag with the conic end. The unit consisting of the 2 catheter and of the 3 drainage bag makes the intermittent clean self-catheterization in a closed system possible.

The device assisting self-catheterization according to the invention as the subject matter of the present patent application can be fixed to 1 grip element (handle) having the same length and diameter like those of the 1 injective syringe used by the second and third group of patients by a fixing clamp.

Using this closed system regularly, (self-catheterization 2-3 per day) only the single use bladder catheter should be changed to a new sterile catheter.

After the bladder drainage and after opening of the 9 spring-grabbing-unit the used 2 catheter will be separated from the 4 tube of the 3 drainage bag and the catheter will be destroyed according to the actual health requirements.

Before the next due self-catheterization a new sterile 2 catheter will be connected to the tube of the 3 urine drainage bag, and after the removal of the protective packaging of the 2 catheter the 1 handle will be fixed on the 2 catheter by the 9 spring-grabbing-unit.

As the replacements are self-evident, separate figure was not made for this subcase.

The light source and the mirror or the camera fixed to the handle can be used also advantageously in this case for the quick and simple location of the urinary meatus (external urethral orifice).

Ebben az esetben is elõnyösen használható a húgycsönyilás gyors és egyszerü felkeresését segitö, a fogantyúra felerösithetö fényforrás és tükör vagy kamera/kamerák.

The device shown in the FIG. 2 is used by the patients with urination problems who need also medication.

For medication the 1 syringe containing drug solution is connected to the 9 three-way tap and the drug solution can be injected into the bladder from the 1 syringe by the proper adjusting of the 9 three-way tap. Before the bladder instillation necessarily an urine drainage should have been preformed through the 4 tube of 3 urine drainage bag into the 3 drainage bag.

For next after changing the position of the 9 three-way tap properly the drug solution can be injected into the bladder through the 2 catheter into the bladder by pressing the 10 piston of the 1 syringe (FIG. 2).

The device shown in the FIGS. 3 and 4 is used by the patients who need solely medication.

In such cases no use of 3 urine drainage bag is needed. According to the FIG. 4 the 2 catheter can also be replaced by the 16 leakage free adapter for injection.

The thickness of the wall of the 16 adapter assures the proper stability and the 17 sealing collar of the adapter assists the leakage-free injection.

By using the device according to the invention these are of operations will be lighted and can be seen by using for example an 8 mirror.

Solutions of the prior art placed the mirror or the lightning independently from the device itself, making the operation difficult and complicated.

By the solution according to our invention the 1 syringe or handle is used as 11 grip element where also the 12 holder element is fixed (FIGS. 5 to 8).

The 6 light source was placed in or on the 12 holder element so that, the 20 light-ray emitted by the 6 light source should light the end of the catheter opposite to the 9 three-way tap and the surrounding of it.

The position of 6 light source can be changed so that, the 20 light-ray should light the end of the catheter and the target-area (the 7 power supply, or the 5 micro-switch or film-switch for switching the light source on-off) the best way for the patient.

The 8 mirror or 13 camera is connected by a flexible self-holding arm as 15 fixing element to the 12 holder part (FIGS. 9 to 11).

So by proper positioning of the 8 mirror or 13 camera and the 6 light source an operation can be performed easily and quickly.

The 13 camera can be replaced by more, advantageously two stereo cameras (FIGS. 8 and 11), and can be supplied by an independent 18 energy source (FIG. 7), or it can be supplied also by the 7 power supply placed on/in the 12 holder element (FIGS. 8, 10 and 11).

In this case all the lines assuring the power supply, and so the whole system can be switched on by the 5 waterproof micro-switch.

The 13 camera consists of 19 communication tool. The wireless communication of the 13 camera could be wifi and/or Bluetooth, and/or infra and/or radio communication.

The formation of the device according to our invention makes possible that, the value of the length of the 2 catheter could be between 150 to 250 mm, advantageously 150 mm or 200 mm (FIG. 1), which makes the use of the device even easier.

Using the longer catheters as earlier the operation could be performed more difficult.

The position of the 1 syringe or 1 handle makes possible for using them as 11 grip element either connected to the medium outlet of the 9 three-way tap or fixed to the 2 catheter by the 9 spring-grabbing-unit. The device can be handled by this method on a proper way.

The 12 holder element is fixed on the 11 grip element advantageously on the 1 syringe or on the 1 handle (FIGS. 5 to 8) as a fast-connecting fixing ring using spring-force or a fixable clamp both fixed by a 14 fixation (FIGS. 9 to 11). The 12 holder element can be also such a flexible horseshoe, where the 14 fixation is unnecessary. This is obvious for expert, so it is not illustrated.

The device according to our invention consists of the following components:

A central, single use 9 three-way tap consisting of Luer Lock connectors, made from plastic.

A short, advantageously 150 mm or 200 mm long single use 2 female catheter made from the usual plastic for medical use, advantageously polyvinyl-chloride (PVC) connected to the 9 three-way tap by Luer Lock connectors.

The 3 urine drainage bag is connected to the 9 three-way tap also by Luer Lock connectors, so an incidental urine flow is inhibited, generating a closed system increasing the sterility of the operation.

As an advantageous solution of the invention in case the device is used solely for bladder drainage the 9 three-way tap is replaced by a connecting 9 spring-grabbing clip-on unit which can be fixed on the 2 catheter and the 2 catheter is connected the usual way directly to the conic end of the 4 tube of the 3 urine drainage bag.

In this case the length of the catheter can be optional, because the 9 grabbing element can be placed at any distance from end of the catheter.

To the medium outlet of the tap an internally threaded 20 ml Luer Lock 11 grip element advantageously 1 syringe or 1 handle is connected.

The 12 holder part expediently made from flexible plastic is connected flexibly or by 14 fixation to the 1 syringe or handle as to 11 grip element.

The expediently circular, plain or concave (enlarger) 8 mirror with a diameter of approx. 100 mm is hold by the 12 holder element approx. 100-200 mm above the urinary meatus.

The 8 mirror could be provided with ball joint or with a 15 flexible self-holding arm, so the 8 mirror can be directed peculiarly to the direction of the urinary meatus.

For lighting the 2 catheter two to four pieces of 6 light forces advantageously LED diodes are fixed on or partly in the two opposite sides of the 12 holder element giving the proper luminous power.

The light forces directed into the direction of the urinary meatus, it can be inhibited that, the shadow of the catheter disturbs locating the target and inserting the catheter properly.

As an alternative solution the 8 mirror is replaced by a small sized 13 camera with high resolution. The optionally zoomable picture can be conveyed wired or wireless (wifi and/or Bluetooth and/or infra and/or radio) by the 13 camera to an optional receiving device which could be smartphone, tablet, notebook, PC monitor, TV monitor, video monitor etc.

By using more, advantageously two 13 camera at the same time more developed three dimensional (3D) video-picture or information could be wirelessly conveyed and the 3 D picture can be visualized by a proper tool like notebook, PC monitor, TV monitor by using 3 D glasses.

As further possibility for visualizing the pictures of a stereo 13 camera, a 3 D headset can be used which assists to locate simpler the right position of the urinary meatus by using virtual reality (VR).

As for a further possibility a smartphone application can be developed for the proper treatment of the diseases by using, registering and analysing the data immediately and continuously, using the Bluetooth connection and the camera by both the doctor and patients.

By changing or removing some components of the modular built medical device developed by us the needs of all three groups of patients can be fulfilled.

The device for using for assisting for treatment of the bladder according to our invention consists of a single use 2 catheter, of a 3 urine drainage bag connected to the catheter assuring closed system, of 1 syringe used also as 11 grip element and of 9 three-way tap bringing all the previous elements together.

With turning the 9 three-way tap into the direction assuring straight flow, the bladder is drained into the 3 drainage bag. After the bladder drainage, by changing the position of the 9 three way tap, the drug solution can be injected from the 1 syringe into the bladder by pressing the 10 piston of the 1 syringe

Using the device at the group of patients where the target is solely the bladder drainage the 1 syringe can be replaced by the 1 handle as 11 grip element and the 9 three-way tap can be replaced by the 9 spring-grabbing clip-on unit in case of using both the syringe or the handle.

The 9 clip-on unit can be connected to the 11 grip element both to 1 syringe or to 1 handle. Necessarily by the use of 1 handle only the 9 grabbing clip-on unit can be used, the three-way tap cannot be used in this case.

At the group of patients where no bladder drainage is needed, because the patient can drain the bladder spontaneous (can be controlled by ultrasound test), but local bladder instillation, medication is needed the device usable only for injection changes as follows:

There is no 3 drainage bag is needed anymore, the device only consists of a 9 three-way tap and where only the 2 short female catheter and the 1 syringe loaded with drug solution is connected. After insertion of the 2 catheter the drug solution can be injected into the bladder through the 9 three-way tap. The 1 syringe serves here also as 11 grip element.

For another alternative solution the 2 catheter can be replaced by a 16 leakage-free adapter for injection, and he drug solution can be injected slowly by using low pressure into the bladder. The special 16 adapter is protected by our own separate patent application making a catheter- and leakage-free and non-invasive bladder instillation possible

By all three groups of patients the 12 holder element consisting of 6 light source of 8 mirror, or of 13 camera or cameras is connected or fixed by 14 fixation to the 11 grip element advantageously to 1 syringe or 1 handle.

Next the 9 three-way tap or 9 clip-on unit, the 2 catheter or the 16 adapter for injection and optionally the 3 urine drainage bag by the 4 tube will be connected to the device.

In case the necessary operation is solely bladder drainage the empty syringe or the handle can be used as 11 grip element. By using the syringe as 11 grip element the 2 catheter for drainage and by the 4 tube the 3 urinary drainage bag is also connected to the 9 three-way tap and the 9 three-way tap is only open to the direction of the 3 urine drainage bag or the 9 tap is replaced by the 9 grabbing clip-on unit the unit will be fixed to the 2 catheter and the catheter is connected directly to the 4 tube of the 3 urine drainage bag.

In case there are both bladder drainage and medication needed, the urine will be drained into the 3 urine drainage bag by adjusting the 9 three-way tap properly so that, the tap will be opened to the direction of 3 urine drainage bag.

After the bladder drainage, by changing the position of the 9 three way tap, the drug solution can be injected from the 1 syringe into the bladder through the 2 catheter by pressing the 10 piston of the 1 syringe slowly and continuously.

In case solely medication is needed the 2 catheter can be replaced also by the 16 leakage-free adapter for injection assuring the free flow between the 9 three-way tap and the 1 syringe.

At the beginning of all three type of treatment the 12 holder element should be fixed to the 11 group element and after the 11 grip element has been connected to the 2 catheter and the 3 urine drainage bag, the 6 light force fixed to the 12 holder element should be switched on by the water-proof 5 micro-switch. The 8 mirror should be directed to the target area. In case 13 camera(s) is(are) used, the camera should be switched on and directed to the target area (in case the switch on is not performed by the 5 micro-switch). Necessarily the devices for visualizing the pictures of the 13 camera(s) should be also switched on.

As the area is now well seen, the 2 catheter or the leakage-free 16 adapter for injection can be directed to the target area for assisting the insertion into the urinary meatus. The insertion position of the 16 adapter is assured by the 17 sealing collar of the adapter and so the necessary local treatment can be performed.

There is a different way of preparing the 11 grip element depending on the three groups of patients:

Concerning the first group, where the target is solely the bladder drainage the 1 syringe or 1 handle is connected to the 2 catheter through the 9 three-way tap or the 9 spring-grabbing clip-on unit and next the 2 catheter is connected either through the 9 three-way tap or directly to the 4 tube of the 3 urine drainage bag, or in the third group where there is no drainage, either the 2 catheter or the leakage-free 16 adapter for injection to be inserted into the urinary meatus will be connected to the medium outlet of the 9 tap.

The advantage of our invention is that, there is no need to transport the patient regularly into the health centre, clinic, for changing the catheter, there is a much lower risk of developing chronic bacterial inflammation, there is no permanent irritation, no sensation of a foreign body, no pain in the urethra.

The complications of inflammation are less frequent and milder.

The regular treatment of the patient does not depend on restraints like the consulting hours of the doctor or the presence of the home assistance, schedule of ambulance transport etc. but depending on the needs of the patient the treatment can be customized.

The syringe is much closer, in an available position for the patient during the local medication of the bladder.

Because of the construction of the device, there is possibility for using the device one handed so by using the other free hand the vulva can be explored properly, and the urinary meatus (external urethral orifice) can be clearly located and sterilized.

By changing or removal of components of the modular medical device according to our invention the need of all three group of patients can be fulfilled.

The handling of the device according to our invention can be trained in a short time.

By the construction a wide spectrum of needs is covered, starting from the simple solution using a mirror through the innovative catheter-free self-medication to the almost futuristic 3 D devices.

This last solution assures that, our device will be modern and usable not only in presence but also in the future for decades.

The use of the device according to our invention assists in care or supply and in improvement of the quality of life, life expectancy of patients who are in serious conditions or disabled, and a significant reduction in the burden of such patients of the risk of complications of catheterizations can be expected.

Learning to handle the device is relatively easy which assures a large scale independency for the patients from the health care and by customizing the treatments according to the needs even the improvement of the therapeutic results can be expected.

### LIST OF REFERENCE NUMBERS

1 syringe or handle
2 catheter
3 urine drainage bag
4 tube of the urine drainage bag
5 micro switch
6 light source
7 power supply
8 mirror
9 three-way tap or spring-grabbing clip-on unit
10 piston
11 grip element
12 holder element
13 camera
14 fixation
15 fixing element
16 leakage-free adapter for injection
17 sealing collar of the adapter

1. Modular built medical device for assisting self-catheterization and instillation of drug-solutions into the bladder in female patients, consisting of a handle or of a syringe, of a catheter, of a three-way tap, of a spring-grabbing clip-on unit of an urine drainage bag, further on of a switchable light source, of a power supply and further on of a mirror characterized by that, instead of or beside the mirror (8) the device consists of a camera (13), and the catheter (2) and the urine drainage bag (3) is connected through the three-way tap (9) or through the spring-grabbing clip-on unit (9) so that, the syringe (1) or handle (1) connected to the medium outlet of the three-way tap (9) or to the spring-grabbing clip-on unit (9) functions also as the grip element (11) of the device, where the holder element (12) is fixed on or in the holder element (12), where also the micro-switch (5), the light source (6) and the power supply (7) can be found, and further on the mirror (8) and/or the camera (13) is fixed directly or with a fixing element (15) to the holder element (12) and the camera (13) is suitable for connection to an optional wired or wireless receiving device.

2. The device according to claim 1 characterized by that the grip element (11) is a syringe (1).

3. The device according to claim 1 characterized by that the grip element (11) is a specially shaped and sized handle (1) so that it can be grasped by hand.

4. The device according to claim 3 characterized by that the handle (1) is hollow with the right thickness of wall for the proper stability.

5. The device according to any of claims 3 and 4 characterized by that the handle (1) is a body closed on the upper side.

6. The device according to any of claims 3 to 5 characterized by that the handle (1) is a cylindrical body.

7. The device according to any of claims 3 to 6 characterized by that the handle (1) is a solid body.

8. The device according to any of claims 3 to 7 characterized by that the handle (1) is a body with the same diameter and length of the syringe (1) according to

9. The device according to any of claims 3 to 8 characterized by that a spring-grabbing clip-on unit (9) is connected-to the handle (1) which can be fixed to the catheter (2) by a releasable clip-on connection.

10. The device according to any of claims 1 to 9 characterized by that instead of or beside the mirror (8) the device consists of a camera (13).

11. The device according to any of claims 1 to 10 characterized by that the full length of the catheter (2) is between 150 mm and 250 mm.

12. The device according to 11 characterized by that the full length of the catheter (2) is 150 mm or 200 mm.

13. The device according to any of claims 1 to 12 characterized by that holder element (12) is fixed as a fast-connecting fixing ring using spring-force or as a fixable clamp both fixed by a releasable fixation (14) or the holder element (12) can be also a flexible horseshoe, where the fixation (14) is unnecessary.

14. The device according to any of claims 1 to 13 characterized by that the fixing element (15) for the mirror (8) and/or the camera (13) is a flexible self-holding arm (15).

15. The device according to any of claims 1 to 14 characterized by that the camera (13) consist of an independent energy force (18) and of a communication device suitable for wireless communication (19).

16. The device according to any of claims 1 to 15 characterized by that the camera (13) is functioning by the power supply (7) placed in or on the holder element (12).

17. The device according to any of claims 10 and 15 to 16 characterized by that the wireless communication of the camera (13) is wifi and/or Bluetooth, and/or infra, and/or radio connection.

18. The device according to any of claims 1 to 17 characterized by that the camera (13) is a stereo camera and/or the device consists of more cameras.

19. The device according to any of claims 1 to 18 characterized by that the picture is conveyed by the wireless connection of the camera (13) to the optional receiving device.

20. The device according to any of claim 19 characterized by that the receiving device is smartphone and/or tablet and/or notebook and/or PC monitor and/or TV monitor and/or video monitor.

21. The device according to any of claims 19 to 20 characterized by that in case the pictures are conveyed through stereo camera (13) the receiving device is a 3 D headset suitable for visualizing 3 D virtual reality.

22. Process for use of modular medical device assisting self-catheterization and self-instillation of drug solutions into the bladder in female patients using the device according to any of claims 1 to 2 and 10 to 21 characterized by that the syringe (1) consisting of the holder element (12), functioning as grip element (11) is taken by one hand and is placed near to the urinary meatus and the end of the catheter (2) or of leakage-free adapter for injection (16) is and the area of them is lighted by the light force (6) fixed on the holder element (12), and next the mirror (8) and/or the camera (13) should be positioned so that, picture of the urinary meatus and of the end of the catheter (2) or of the adapter (16) could be seen in the mirror (8) or in the camera (13) and so the catheter (2) or the leakage-free adapter for injection (16) can be inserted in full length into the urethra and for next the bladder drainage and/or the bladder self-instillation of drug solution can be performed.

23. Process for use of modular medical device assisting self-catheterization and self-instillation of drug solutions into the bladder in female patients using the device according to any of claims 1 and 3 to 21 characterized by that the handle (1) consisting of the holder element (12), functioning as grip element (11) is taken by one hand and is placed near to the urinary meatus and the end of the catheter (2) and the area of them is lighted by the light force (6) fixed on the holder element (12), and next the mirror (8) and/or the camera (13) should be positioned so that, picture of the urinary meatus and of the end of the catheter (2) could be seen in the mirror (8) or in the camera (13) and so the catheter (2) can be inserted in full length into the urethra and for next the bladder drainage can be performed.

24. The process according to any of claims 20 to 23 characterized by that the pictures are conveyed through stereo camera (13) using 3 d glasses or 3 D head set for viewing the pictures.
